(19) United States
(12) Patent Application Publication
(10) Pub. No.: US 2022/0254275 A1
(43) Pub. Date: Aug. 11, 2022

(54) SYSTEM AND A METHOD FOR PROVIDING POSITIVE REINFORCEMENT ON APPROPRIATE FOOD ITEMS

(71) Applicant: Krishna Sarathy Venugopal, The Woodlands, TX (US)

(72) Inventors: Krishna Sarathy Venugopal, The Woodlands, TX (US); Raghavan Veera Venugopal, The Woodlands, TX (US)

(21) Appl. No.: 17/169,824

(22) Filed: Feb. 8, 2021

(51) Int. Cl. G09B 19/00; G16H 20/60; G06F 16/901; G06F 16/906; G06F 16/9035

(52) U.S. Cl. CPC G09B 19/0092; G16H 20/60; G06F 16/9035; G06F 16/906; G06F 16/901

(57) ABSTRACT

Disclosed is a system for providing positive reinforcement on appropriate food items. The system includes a storage unit, a graphical user interface, a food central database, a pillar database and a processing unit. The storage unit stores plurality of modules, the food central database stores type of nutrient and nutrient value associated with each food item. The pillar database categorizes the nutrients and the nutrient value of each food item into either a growth pillar, an energy pillar, an immunity pillar, and a detoxification pillar. The plurality of modules includes a growth module, an energy module, an immunity score and a detoxification module coupled to the pillar database to evaluate a growth score, an energy score, a detoxification module respectively from the growth pillar, the energy score, the immunity score, and the detoxification score for each food item. The growth score, the energy score, the immunity score and the detoxification score is evaluated from the growth related nutrients value associated with each nutrient.

### BACKGROUND OF THE INVENTION

### 1. FIELD OF THE INVENTION

The present invention generally relates to a system and a method for suggesting food items, and more specifically relates to a method and a system for calculating pillar scores associated with each food items to provide positive reinforcement.

### 2. DESCRIPTION OF RELATED ART

Lifestyle habits such as eating, exercising, and sleeping have significant effects on physical condition of a human. Among these lifestyle habits, eating is especially important. Since physical condition is reflected on the appearance of the human, eating healthy is important also from a viewpoint of appearance. Further, eating is also enjoyment for the human, and it is possible for the human to maintain his/her health without being bored by properly devising the selection of food and menu.

Making healthier food choices can help prevent non-communicable diseases such as obesity, cardiovascular disease, diabetes, and some cancers. Guidelines, like the Dietary Guidelines for Americans, provide general population recommendations for healthful eating. Such guidelines can drive content for nutrition labels, health claims, nutrition education, menu planning, as well as marketing and advertising on food products. However, these generalized guidelines are not designed to provide personalized nutrition recommendations at the macro- or micronutrient level.

Firstly, measurement of food happens only through calories so awareness revolves around calories and the idea that the number of calories someone eats dictates whether they are healthy or not. The second problem is with the selections and that the choices of food are not personalized. For example, the classification of someone eating a certain type of food, like a vegetarian or vegan, is overly broad and does not say enough about the actual food itself. Also, food that is good for one person is not necessarily as good for another person.

The third problem with today's method of food selection and awareness connects with the previous problem with the classification of food. Close-loop feedback is not available to show whether the individual's personalized food choices are making an expected impact on their body, mind, and intellect. Instead, these loops are often forgotten, and in most cases, not even looked at as a follow-up on a person's health.

Substantial efforts have been made to quantify and track the impact of particular consumables, such as ingredients, foods, meals, or diets, on the overall health of individuals. For example, every five years since 1980, the United States Department of Agriculture (USDA) along with the Department of Health and Human Services (HHS) releases so-called Dietary Guidelines for Americans.

The USDA states that these Guidelines provide advice about consuming fewer calories, making informed food choices, and being physically active to attain and is maintain a healthy weight. (http://www.cnpp.usda.gov/DietaryGuidelines). With this background, in recent years, there have been many technologies for supporting the selection of the food and menu widespread in the world.

However, the existing methods and systems fails to compute each nutrients and their nutrient value for each food item. Further, the existing methods and systems provide overall score and doesn't bifurcate the nutrients into important pillars of health. Each food item should be graded on the basis of growth, immunity, energy, and detoxification. Therefore, there is a need of a method and a system for calculating pillar scores associated with each food items to provide positive reinforcement.

### SUMMARY OF THE INVENTION

In accordance with teachings of the present invention, a system for providing positive reinforcement on appropriate food items is provided.

An object of the present invention is to provide a system having a storage unit, a graphical user interface, a food central database, a pillar database, and a processing unit. The storage unit stores plurality of modules, the graphical user interface displays processed plurality of modules, the food central database stores type of nutrient and nutrient value associated with each food item.

The pillar database is coupled to the food central database for categorizing the nutrients and the nutrient value of each food item into either a growth pillar, an energy pillar, an immunity pillar, and a detoxification pillar. The processing unit is operably coupled to the food central database, the pillar database, the graphical user interface, and to the storage unit to process the plurality of modules.

The plurality of modules include a growth module coupled to the pillar database to evaluate a growth score from the growth pillar for each food item. The growth score is evaluated from the growth related nutrients value associated with each nutrient. The energy module is coupled to the pillar database to evaluate an energy score from the energy pillar for each food item.

The energy score is evaluated from the energy related nutrients value associated with each nutrient and the immunity module coupled to the pillar database to evaluate an immunity score from the immunity pillar for each food item, wherein the immunity score is evaluated from the immunity related nutrients value associated with each nutrient.

Another object of the present invention is to provide the plurality of modules with a detoxification module being coupled to the pillar database to evaluate a detoxification score from the detoxification pillar for each food item, wherein the detoxification score is evaluated from the detoxification related nutrients value associated with each nutrient. Further, the nutrient values are calculated from a pre-defined serving value.

Another object of the present invention is to provide the plurality of modules having a food interest module for allowing registered users to input a list of food items through the graphical user interface, and a food descriptive module for calculating and providing score for each pillar based on the food items received in the food interest module.

Another object of the present invention is to provide the plurality of modules having a food descriptive module for calculating and providing score for each pillar based on the food items received in the food interest module, and a food predictive module for evaluating and automatically suggesting the registered users to eat specific food items to improve on the growth score, the energy score, and the immunity score, and a food prescriptive module for evaluating and automatically suggesting the food items to score better on the growth score, the energy score, and the immunity score based on the details received from the registered users.

Another object of the present invention is to provide the plurality of modules having a search module to allow the registered user to search nutrients and nutrient value associated with each food item and a social media module to link social media platforms is with the plurality of modules.

Another object of the present invention is to provide a non-transitory computer-readable medium storing computer-executable instructions that, when executed by at least one processing unit of a computer, are configured to cause the computer to implement the system.

While a number of features are described herein with respect to embodiments of the inventions; features described with respect to a given embodiment also may be employed in connection with other embodiments. The following description and the annexed drawings set forth certain illustrative embodiments of the inventions. These embodiments are indicative, however, of but a few of the various ways in which the principles of the inventions may be employed. Other objects, advantages, and novel features according to aspects of the inventions will become apparent from the following detailed description when considered in conjunction with the drawings.

### BRIEF DESCRIPTION OF DRAWINGS

The annexed drawings, which are not necessarily to scale, show various aspects of the inventions in which similar reference numerals are used to indicate the same or similar parts in the various views.

FIG. 1 illustrates a block diagram of a system for providing positive reinforcement on appropriate food items in accordance with an embodiment of the present invention;

FIG. 2 illustrates a block diagram of a pillar database in accordance with an exemplary embodiment of the present invention;

FIG. 3 illustrates a block diagram showcasing a growth score, an energy score, an immunity score and a detoxification score in accordance with an exemplary embodiment of the present invention;

FIG. 4 illustrates a block diagram for a registration module in accordance with an exemplary embodiment of the present invention;

FIG. 5 illustrates a screenshot showing of a graphical user interface of a food interest module in accordance with an exemplary embodiment of the present invention;

FIG. 6 illustrates a screenshot showing of a graphical user interface of a food descriptive module in accordance with an exemplary embodiment of the present invention;

FIG. 7 illustrates a table for showing a food predictive module in accordance with an exemplary embodiment of the present invention;

FIG. 8 illustrates a table for showing a food prescriptive module in accordance with an exemplary embodiment of the present invention;

FIG. 9 illustrates a screenshot showing of a graphical user interface of a search module in accordance with an exemplary embodiment of the present invention; and

FIG. 10 illustrates a flowchart for showing a method for providing positive reinforcement on appropriate food items in accordance with an embodiment of the present invention.

### DETAILED DESCRIPTION OF DRAWINGS

The present disclosure is now described in detail with reference to the drawings. In the drawings, each element with a reference number is similar to other elements with the same reference number independent of any letter designation following the reference number. In the text, a reference number with a specific letter designation following the reference number refers to the specific element with the number and letter designation and a reference number without a specific letter designation refers to all elements with the same reference number independent of any letter designation following the reference number in the drawings.

The present disclosure provides in one embodiment a computer-implemented method for providing positive reinforcement on appropriate food items is stored on a non-transitory computer readable storage medium. The method is performed through plurality of modules at least in part by a processing unit and the plurality of modules are stored in a storage unit.

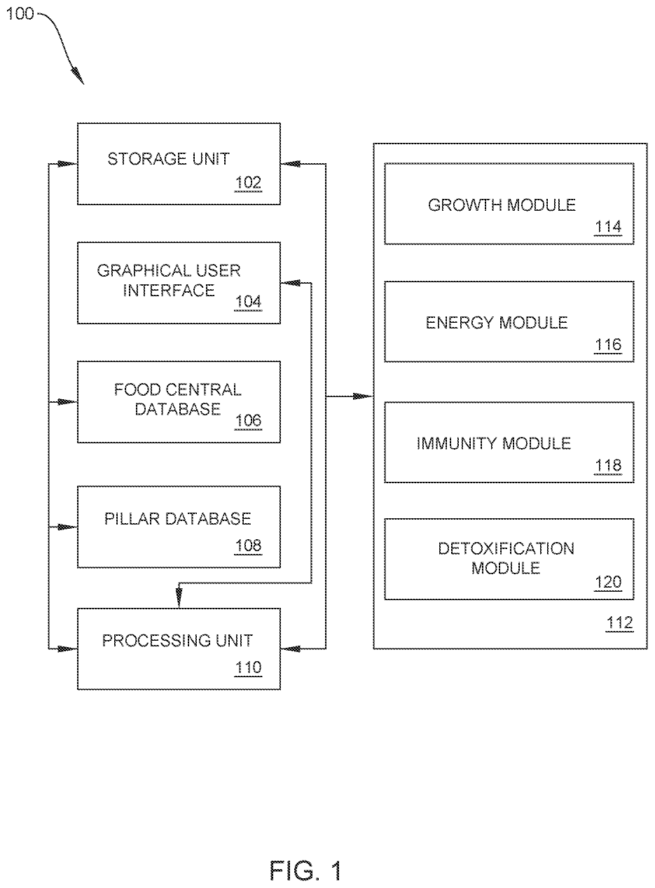

FIG. 1 illustrates a block diagram of a system 100 for providing positive reinforcement on appropriate food items in accordance with an embodiment of the present invention. The system 100 include a storage unit 102, a graphical user interface 104, a food central database 106, a pillar database 108, and a processing unit 110.

The storage unit 102 stores plurality of modules 112. The graphical user interface 104 displays processed plurality of modules 112. The food central database 106 stores type of nutrients and their nutrient value associated with each food item. The pillar database 108 is coupled to the food central database 106 for categorizing the nutrients and the nutrient value of each food item into a growth pillar or an energy pillar or an immunity pillar or a detoxification pillar or in combination.

Examples of the storage unit 102, the food central database 106 and the pillar database 108 include but not limited to SQL Server, Oracle Database, Sybase, Informix, and MySQL. Examples of the graphical user interface 104 include but not limited to LCD, LED, OLED and other similar display items. It would be readily apparent to those skilled in the art that various types of the storage unit 102 and the graphical user interface 104 may be envisioned without deviating from the scope of the present invention.

The processing unit 110 is operably coupled to the food central database 106, the pillar database 108, the graphical user interface 104 and to the storage unit 102 to process the plurality of modules 112. The plurality of modules 112 include a growth module 114, an energy module 116, an immunity module 118, and a detoxification module 120. The growth module 114 is coupled to the pillar database 108 to evaluate a growth score from the growth pillar for each food item. The growth score is evaluated from the growth related nutrients value associated with each nutrient. For example, the growth score can be calculated by extracting nutrient values from food that promote growth of a human user. In some examples, the processing unit 110 may be a central processing unit (CPU), a semiconductor based microprocessor, and/or other hardware devices suitable for retrieval and execution of instructions stored in non-transitory computer readable medium (e.g., plurality of modules 112, etc.). In some examples, the food central database 106 can be a non-transitory computer readable medium. As used herein, a non-transitory computer readable medium may be electronic, magnetic, optical, or other physical storage device that stores executable instructions. For example, a non-transitory computer readable medium may be a Random Access Memory (RAM), an Electrically-Erasable Programmable Read-Only Memory (EEPROM), a storage drive, and the like.

The energy module 116 is coupled to the pillar database 108 to evaluate an energy score from the energy pillar for each food item. The energy score is evaluated from the energy related nutrients value associated with each nutrient. For example, the energy score can be calculated by extracting nutrient values from food that promote energy of a human user. The immunity module 118 is coupled to the pillar database 108 to evaluate an immunity score from the immunity pillar for each food item. The immunity score is evaluated from the immunity related nutrients value associated with each nutrient. For example, the immunity score can be calculated by extracting nutrient values from food that promote immunity of a human user.

In another embodiment of the present invention, the plurality of modules 112 include a detoxification module 120 coupled to the pillar database 108 to evaluate a detoxification score from the detoxification pillar for each food item. The detoxification score is evaluated from the detoxification related nutrients value associated with each nutrient.

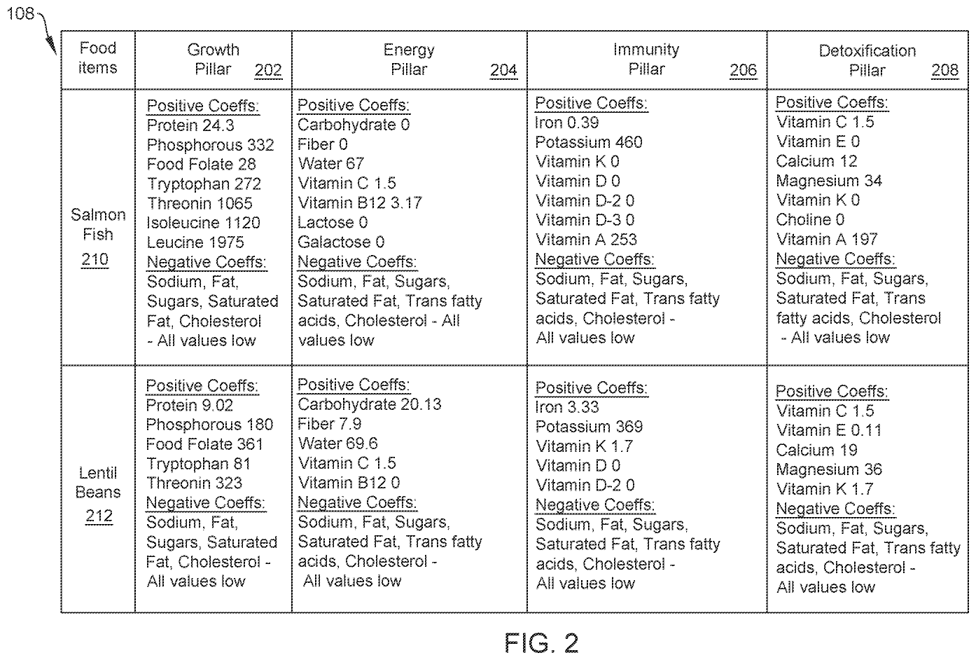

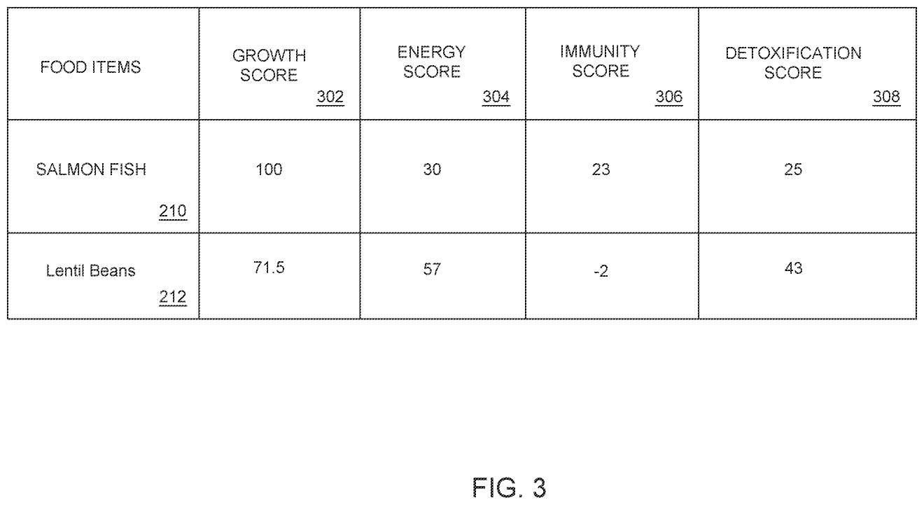

The pillar database 108 is explained in detail in conjunction with FIG. 2 of the present invention, The growth module 114, the energy module 116, the immunity module 118 and the detoxification module 120 is explained in detail in conjunction with FIG. 3 of the present invention.

FIG. 2 illustrates a block diagram of a pillar database 108 in accordance with an exemplary embodiment of the present invention. The pillar database 108 categorize the nutrients and nutrient value of each food item into a growth pillar 202, an energy pillar 204, an immunity pillar 206 and a detoxification pillar 208.

For exemplary purposes, the food items salmon fish 210 and lentil beans 212 are categorized into nutrients and nutrient value. The nutrients of salmon fish 210 and the lentil beans 212 is categorized into the growth pillar 202, the energy pillar 204, the immunity pillar 206, and the detoxification pillar 208.

The growth positive nutrients and the nutrient value for salmon fish 210 are Protein 24.3, Phosphorous 332, Food Folate 28, Tryptophan 272, Threonin 1065, Isoleucine 1120, Leucine 1975, etc. Whereas the growth negative nutrients and the nutrient value for salmon fish 210 are Sodium, Fat, Sugars, Saturated Fat, Cholesterol—All values low.

Similarly, the energy positive nutrients and the nutrient value for salmon fish 210 are Carbohydrate 0, Fiber 0, Water 67, Vitamin C 1.5, Vitamin B12 3.17, Lactose 0, Galactose 0, etc. Whereas the energy negative nutrients and the nutrient value for salmon fish 210 are Sodium, Fat, Sugars, Saturated Fat, Cholesterol—All values low.

The growth positive nutrients and the nutrient value for lentils beans 212 are Protein 9.02, Phosphorous 180, Food Folate 361, Tryptophan 81, Threonin 323, etc. Whereas the growth negative nutrients and the nutrient value for salmon fish 210 are Sodium, Fat, Sugars, Saturated Fat, Cholesterol—All values low.

Similarly, the energy positive nutrients and the nutrient value for lentils beans 212 are Carbohydrate 20.13, Fiber 7.9, Water 69.6, Vitamin C 1.5, Vitamin B12 0, etc. Whereas the energy negative nutrients and the nutrient value for salmon fish 210 are Sodium, Fat, Sugars, Saturated Fat, Cholesterol—All values low.

Similarly, the positive and negative coefficients for the immunity pillar 206 and the detoxification pillar 208 are categorized for the salmon fish 210 and the lentil beans 212. It would be readily apparent to those skilled in the art that various food items may be categorized into the growth pillar 202, the energy pillar 204, the immunity pillar 206, and the detoxification pillar 208 without deviating from the scope of the present invention.

In another embodiment of the present invention, the nutrients are classified into unique codes such as the following:

D—Calories
E—Fat (g)
F—Protein (g)
G—Carbohydrate (g)
H—Sugars (g)
I—Fiber (g)
J—Cholesterol (mg)
K—Saturated Fats (g)
L—Calcium (mg)
M—Iron Fe (mg)
N—Potassium, K (mg)
O—Magnesium (mg)
P—Vitamin A, IU (IU)
Q—Vitamin A, RAE (mcg)
R—Vitamin C (mg)
S—Vitamin B-12 (mcg)
T—Vitamin D (mg)
U—Vitamin E (Alpha-Tocopherol) (mg)
V—Added Sugar (g)
W—Net-Carbs (g)
X—Water (g)
Y—Omega 3s (mg)
Z—Omega 6s (mg)
AA—PRAL score (g)
AB—Trans Fatty Acids (g)
AC—Soluble Fiber (g)
AD—Insoluble Fiber (g)
AE—Sucrose (g)
AF—Glucose (Dextrose) (g)
AG—Fructose (g)
AH—Lactose (g) Galactose (g) Starch (g)
AI—Maltose (g)
AJ—Galactose (g)
AK—Starch (g)
AL—Total sugar alcohols (g)
AM—Phosphorus, P (mg)
AN—Sodium (mg)
AO—Zinc, Zn (mg)
AP—Copper, Cu (mg)
AQ—Manganese (mg)
AR—Selenium, Se (mcg)
AS—Fluoride, F (mcg)
AT—Molybdenum (mcg)
AU—Chlorine (mg)
AV—Thiamin (B1) (mg)
AW—Riboflavin (B2) (mg)
AX—Niacin (B3) (mg)
AY—Pantothenic acid (B5) (mg)
AZ—Vitamin B6 (mg)
BA—Biotin (B7) (mg)
BB—Folate (B9) (mg)
BC—Folic acid (mcg)
BD—Food Folate (mcg)
BE—Folate DFE (mcg)
BF—Choline (mg)
BG—Betaine (mg)
BH—Retinol (mcg)
BI—Carotene, beta (mcg)
BJ—Carotene, alpha (mcg)
BK—Lycopene (mcg)
BL—Lutein + Zeaxanthin (mcg)
BM—Vitamin D2 (ergocalciferol) (mcg)
BN—Vitamin D3 (cholecalciferol) (mcg)
BO—Vitamin D (IU) (IU)
BP—Vitamin K (mcg)
BQ—Dihydrophylloquinone (mcg)
BR—Menaquinone-4 (mcg)
BS—Fatty acids, total monounsaturated (mg)
BT—Fatty acids, total polyunsaturated (mg)
BU—18:3 n-3 c, c, c (ALA) (mg)
BV—20:5 n-3 (ERA) (mg)
BW—22:5 n-3 (DPA) (mg)
BX—22:6 n-3 (DHA) (mg)
BY—Tryptophan (mg)
BZ—Threonine (mg)
CA—Isoleucine (mg)
CB—Leucine (mg)
CC—Lysine (mg)
CD—Methionine (mg)
CE—Cystine (mg)
CF—Phenylalanine (mg)
CG—Tyrosine (mg)
CH—Valine (mg)
CI—Arginine (mg)
CJ—Histidine (mg)
CK—Alanine (mg)
CL—Aspartic acid (mg)
CM—Glutamic acid (mg)
CN—Glycine
CO—Proline (mg)
CP—Serine (mg)
CQ—Hydroxyproline (mg)
CR—Alcohol (g)
CS—Caffeine (mg)

The above codes assist in calculating the growth score, the energy score, the immunity score and the detoxification score. The calculation of the growth score, the energy score, the immunity score and the detoxification score is explained in detail in conjunction with FIG. 3 of the present invention.

FIG. 3 illustrates a block diagram showcasing a growth score 302, an energy score 304, an immunity score 306, and a detoxification score 308 in accordance with an exemplary embodiment of the present invention. The salmon fish 210 has the growth score 302 of ‘100’, the energy score 304 of ‘30’ and the immunity score 306 of ‘23’. Further, the lentil beans 212 has the growth score 302 of ‘71.5’, the energy score 304 of ‘57’ and the immunity score 306 of ‘−2’.

The growth score 302, the energy score 304, the immunity score 306, and the detoxification score 308 is calculated by using the nutrients and nutrient value provided in the growth pillar 202, the energy pillar 204 the immunity pillar 206, and the detoxification pillar 208, respectively.

In an exemplary embodiment of the present invention, the growth score 302 is calculated using the following calculation:

IF(AM210>50,5, IF(AM210>25,3, IF(AM210>15,1,0))) IF(F210>9,30, (IF(F210>7,20, (IF(F210>5,10,0)))))−IF(C210<>“Beans and Lentils”, IF(AN210>770,10,0),0)−IF(C210<>“Beans and Lentis”, IF(H210>10,15,0),0)−IF(C210<>“Beans and Lentis”, IF(E210>25,15, IF(E210>17,10,0)),0)−IF(J210>1000,10, IF(J210>500,7, IF(J210>300,3,0)))−IF(K210>15,10, IF(K210>10,7, IF(K210>3,3,0)))+IF(BC210>500,5, IF(BC210>250,2.5, (IF(BC210>100,1,0)))))+IF(BD210>500,5, (IF(BD210>250, 2.5, (IF(BD210>100,1,0)+IF(BE210>500,5, (IF(BE210>250, 2.5,(IF(BE210>100,1,0)))))))))+IF(BY210>100,5, (IF(BY210>50,2.5, (IF(BY210>25,1,0)))))+IF(BZ210>300,5, (IF(BZ210>200,2.5, (IF(BZ210>100,1,0)))))+IF(CA210>400,5, (IF(CA210>200,2.5, (IF(CA210>100,1,0)))))+IF(CB210>1400,5,(IF(CB210>700,2.5,(IF(CB210>350,1,0))))) +IF(CC210>1300,5, (IF(CC210>700,2.5, (IF(CC210>350,1,0)))))+IF(CD210>100,5, (IF(CD210>50,2.5, (IF(CD210>25,1,0)))))+IF(CE210>150,5, (IF(CE210>100,2.5, (IF(CE210>50,1,0)))))+IF(CF210>700,5, (IF(CF210>350,2.5, (IF(CF210>100,1,0)))))+IF(CG210>400,5,(IF(CG210>200,2.5, (IF(CG210>100,1,0)))))+IF(CH210>500,5, (IF(CH210>250,2.5,(IF(CH210>100,1,0)))))+IF(C1210>500,5,(IF(CH210>200,2.5, (IF(CI210>100,10)))))+IF(CJ210>400,5, (IF(CJ210>200,2.5. (IF(CJ210>100,1,0)))))+IF(CK210>400,5, (IF(CK210>200,2.5,(IF(CK210>100,1,0)))))

Similarly, the calculations for the energy score 304, the immunity score 306 and the detoxification score 308 are created. Generally, the calculation sums the individual growth/energy/immunity/detoxification scores of all food categories depending on whether they have been consumed in that day and divides the total growth/energy/immunity/detoxification aggregate by the count of applicable growth/eneray/immunity/detoxification category foods for that segment to arrive at the segment-wise growth/energy/immunity/detoxification score.

It would be readily apparent to those skilled in the art that many possible variations of the calculations may be envisioned to calculate the growth score 302, the energy score 304, the immunity score 306, and the detoxification score 308 without deviating from the scope of the present invention.

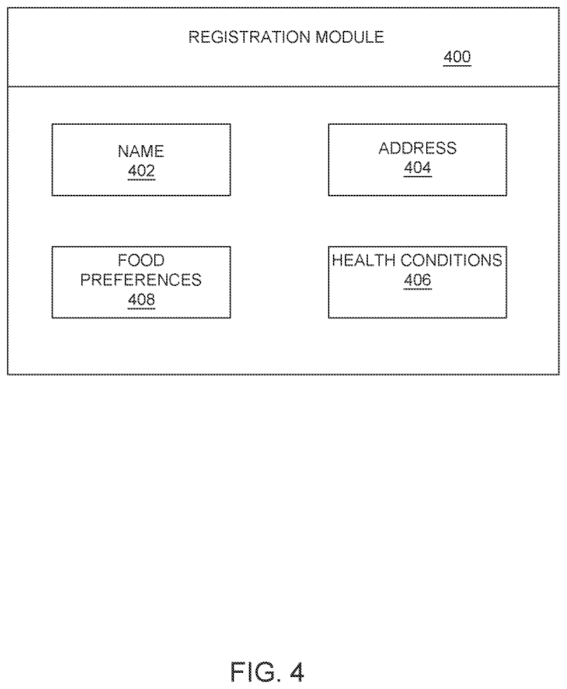

FIG. 4 illustrates a block diagram for a registration module 400 in accordance with an exemplary embodiment of the present invention. The registration module 400 registers users on receiving personal details. Examples of the personal details include but not limited to a name 402, an address 404, health conditions 406, and a food preference 408. It would be readily apparent to those skilled in the art that various personal details such as social media accounts may be received without deviating from the scope of the present invention.

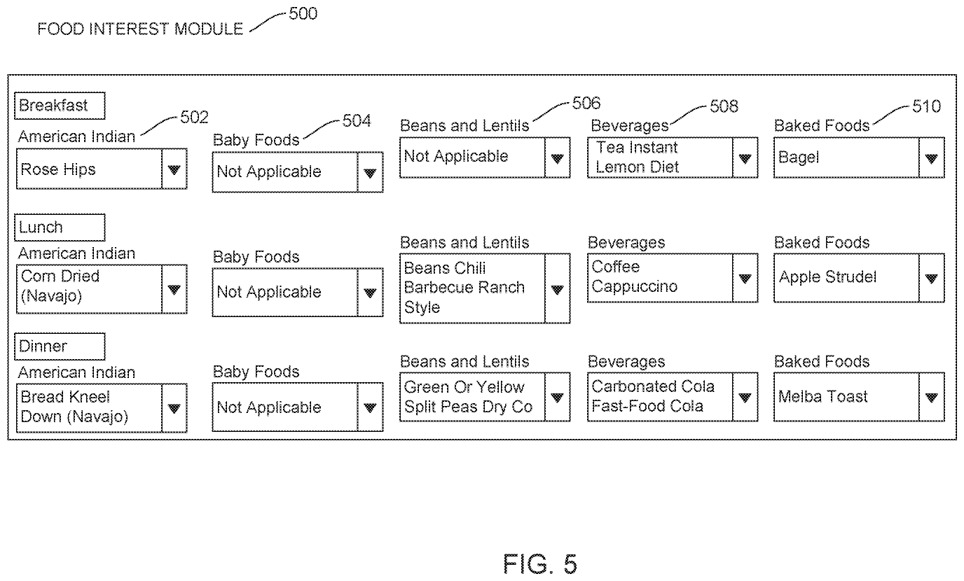

FIG. 5 illustrates a screenshot showing of a graphical user interface of a food interest module 500 in accordance with an exemplary embodiment of the present invention. The food interest module 500 allows registered users to input a list of food items through the graphical user interface.

In an exemplary embodiment of the present invention, the food items are displayed in various categories such as but not limited to American Food 502, Baby Foods 504, Beans and Lentils 506, Beverages 508, and Baked Foods 510, etc. Other categories include but not limited to dairy & Egg Products, Fast Foods, Fats & Oils, Fish, Fruits, Grains & Pasta, Meat, etc.

The categories are extracted from the food central database. The food central database is linked to US Department of Agriculture (USDA) and categorizes around 15000 food items into 19 different categories. The USDA is linked with the Food Data Central (FDC) of the USA, It would be readily apparent to those skilled in the art that various categories and food items may be added into the food central database without deviating from the scope of the present invention.

The user selects the food items in one or more categories for the breakfast, lunch, and dinner. For exemplary purposes, for the breakfast, the user selects Rose Hips for American Indian 502, not applicable for Baby Foods 504, not applicable for Beans and Lentils 506, Tea Instant Lemon Diet for Beverages 508, Bagel for Baked Foods 510. For the Lunch, the user selects Corn Dried (Navajo) for American Indian 502, not applicable for Baby Foods 504, Beans Chili Barbecue Ranch Style for Beans and Lentils 506, Coffee for Beverages 508, and Apple Strudel for Baked Foods.

Similarly, the user selects food for the Dinner. The similar process is followed by the user for other days. It would be readily apparent to those skilled in the art that various types of food items may be selected by the user in the food interest module 500 without deviating from the scope of the present invention.

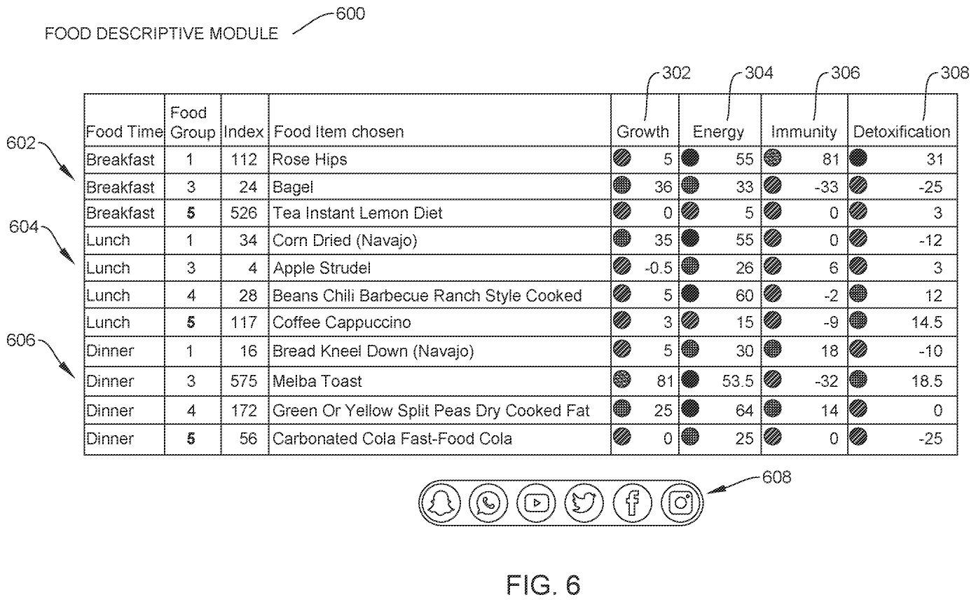

FIG. 6 illustrates a screenshot showing of a graphical user interface of a food descriptive module 600 in accordance with an exemplary embodiment of the present invention. The food descriptive module 600 is coupled with the food interest module (500, shown in FIG. 5) for calculating and displaying score for each pillar based on the food items received in the food interest module (500, shown in FIG. 5).

For exemplary purposes as shown in FIG. 6, the growth score 302 at the time of breakfast 602 for Rose Hips, Bagel and Tea Instant Lemon Diet is ‘5’, ‘36’ and ‘0’ respectively. The energy score 304 at the time of breakfast 602 for Rose Hips, Bagel and Tea Instant Lemon Diet is ‘55’, ‘33’, ‘5’, respectively. The immunity score 306 at the time of breakfast 602 for Rose Hips, Bagel and Tea Instant Lemon Diet is ‘81’, ‘−33’, ‘0’ respectively. The detoxification score 308 at the time of breakfast 602 for Rose Hips, Bagel and Tea Instant Lemon Diet is ‘31’, ‘3’, respectively.

Similarly, the growth score 302, the energy score 304, the immunity score 306, and the detoxification score 308 is calculated for the lunch 604 and the dinner 606. In an exemplary embodiment of the present invention, the growth score 302, the energy score 304, the immunity score 306, and the detoxification score 308 is calculated using the calculation explained in FIG. 3 of the present invention.

In another preferred embodiment of the present invention, the system 100 includes a social media module 608 allows the registered users to communicate with social media platforms. Examples of the social media platforms include but not limited to Snapchat™, WhatsApp™, YouTube™, Twitter™, Facebook™, and Instagram™, etc.

In a preferred embodiment of the present invention, the registered users are able to post their diets, score pattern, etc. on the social media platforms. Further, the registered users may create any competition/challenge on the social medial platforms. It would be readily apparent to those skilled in the art that various forms of social media post and avatars of users may be created without deviating from the scope of the present invention.

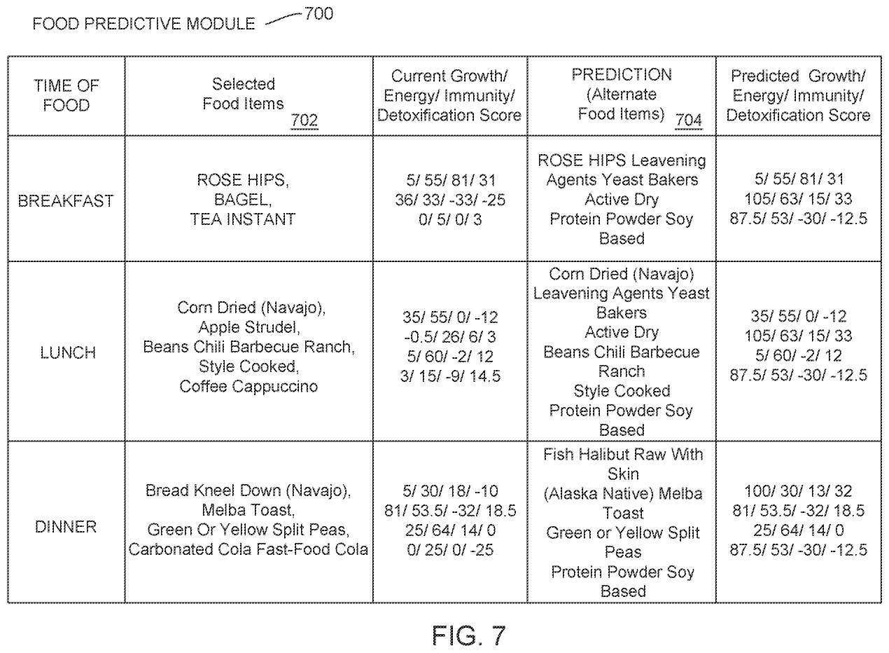

FIG. 7 illustrates a table for showing a food predictive module 700 in accordance with an exemplary embodiment of the present invention. The food predictive module 700 for evaluating and automatically suggesting the registered users to eat specific food items 702 to improve on the growth score, the energy score, the immunity score, and the detoxification module.

The food prediction module 700 suggests/predicts 704 alternative food items within the same category. The food predictive module 700 suggests alternative food items that are high on nutrient value for a particular pillar and/or overall in comparison to the selected food items. For exemplary purposes as shown in FIG. 7, the food predictive module 700 suggests ‘leavening agents yeast bakers active dry’ instead of bagel to get more nutrient value. The ‘leavening agents yeast bakers active dry’ scores high on the growth score and the energy score compared to the bagel.

Similarly, the food predictive module 700 suggests alternative food items for the breakfast, the lunch, and the dinner. It would be readily apparent to those skilled in the art that various calculations may be envisioned to identify alternative food items that scores high on the growth score, the energy score, the immunity score, and the detoxification module without deviating from the scope of the present invention.

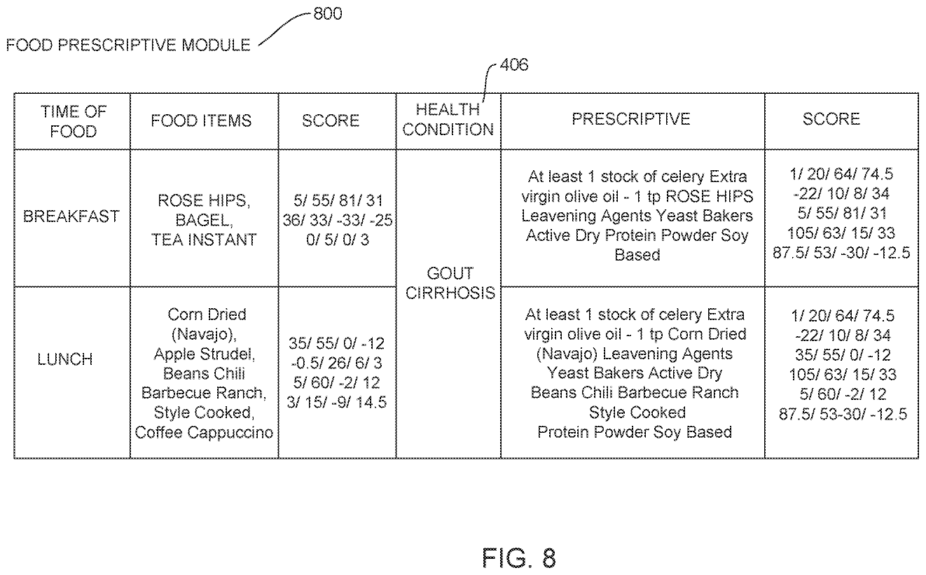

FIG. 8 illustrates a table for showing a food prescriptive module 800 in accordance with an exemplary embodiment of the present invention. The food prescriptive module 800 for evaluating and automatically suggesting the food items to score better on the growth score, the energy score, and the immunity score based on the health conditions 406 received from the registered users. The food prescriptive module 800 is coupled to the food interest module 500.

For exemplary purposes as shown in the FIG. 8, the health conditions 406 are gout and cirrhosis. Thus, in addition to the food items suggested by predictive module (700, shown in FIG. 7), the food prescriptive module 800 recommends the user to add ‘at least 1 stock of celery’ and ‘extra virgin olive oil—1tp’ to increase immunity and detoxification score before every meal. In this way, the health conditions 406 can provide food recommendations based on the registered user's health conditions as well as the current diet of the registered user.

It would be readily apparent to those skilled in the art that the food prescriptive module 800 may be programmed to incorporate various health conditions and to suggest food items to assist in overcoming the health conditions, without deviating from the scope of the present invention.

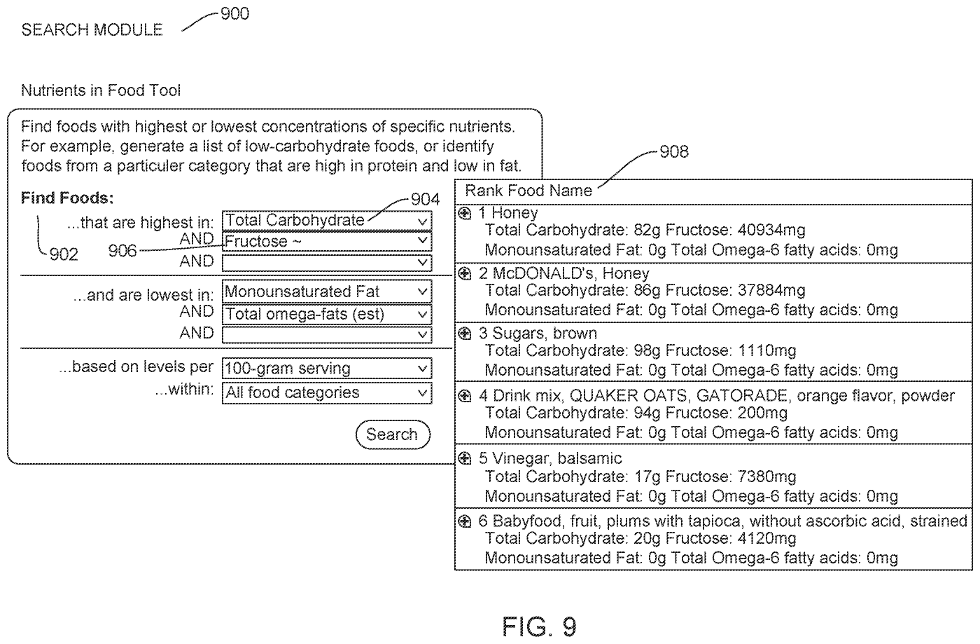

FIG. 9 illustrates a screenshot showing of a graphical user interface of a search module 900 in accordance with an exemplary embodiment of the present invention. The search module 900 allows the registered user to search nutrients and nutrient value associated with each food item.

For exemplary purposes as shown, users input requests in find food 902 such as ‘that are highest in’: ‘Total Carbohydrate’ 904, and ‘Fructose’ 906. The search module 900 then search the pillar database to retrieve the food items 908 based on the inputs received from the user. In this way, a registered user can search for food that includes nutrients that are important to the registered user, suggested for a particular diet, and/or prescribed by a medical professional.

The food items 908 displays the list of carbohydrate and fructose based food items i.e. Honey. McDonald's Honey, Sugars Brown, Drink Mix, Vinegar Balsamic etc. It would be readily apparent to those skilled in the art that various categories of food items may be input by the user to retrieve associated food items. In this way, a user can identify how the food items will affect the different pillars as described herein.

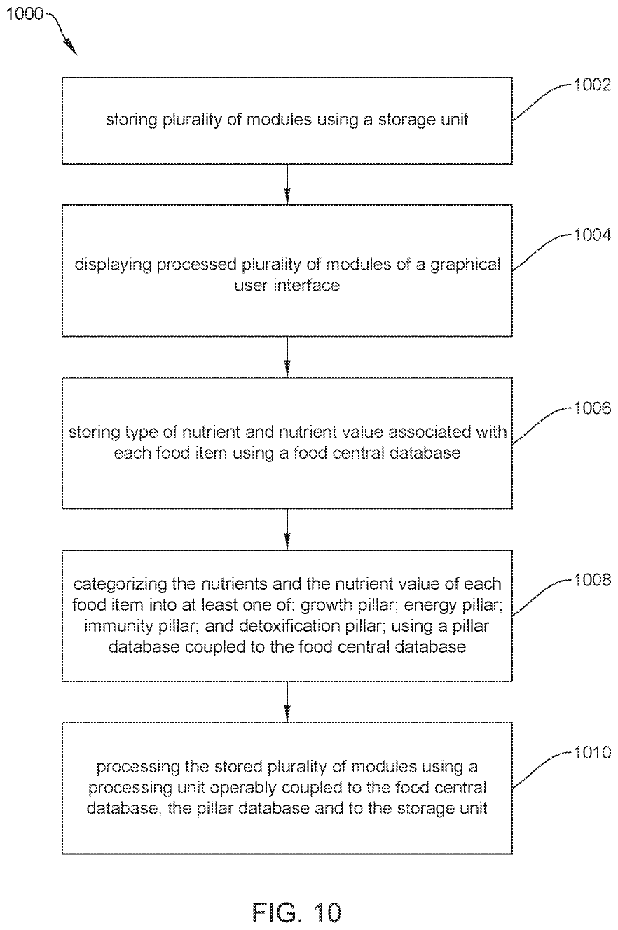

FIG. 10 illustrates a flowchart for showing a method 1000 for providing positive reinforcement on appropriate food items in accordance with an embodiment of the present invention. The method 1000 initiates with a step 1002 of storing plurality of modules using a storage unit, followed by a step 1004 of displaying processed plurality of modules of a graphical user interface, followed by a step 1006 of storing type of nutrient and nutrient value associated with each food item using a food central database.

The step 1006 is then followed by a step 1008 of categorizing the nutrients and the nutrient value of each food item into at least one of: growth pillar; energy pillar; immunity pillar; and detoxification pillar; using a pillar database coupled to the food central database, and followed by a step 1010 of processing the stored plurality of modules using a processing unit operably coupled to the food central database, the pillar database and to the storage unit. The steps 1002 to 1010 are explained in detailed in conjunction with FIG. 1 to FIG. 9 of the present invention.

The present invention offers various advantages such as providing a digital platform to evaluate nutrition score based on the pillar factor such as a growth factor, the energy factor, the immunity factor, and the detoxification factor. Further the present invention suggests food items to score high on each of the pillar score. Further the present invention helps the user to have proper nutrition based upon their preferred food choices.

It should be appreciated that many of the elements discussed in this specification may be implemented in a hardware circuit(s), a circuitry executing software code or instructions which are encoded within computer readable media accessible to the circuitry, or a combination of a hardware circuit(s) and a circuitry or control block of an integrated circuit executing machine readable code encoded within a computer readable media. As such, the term circuit, module, server, application, or other equivalent description of an element as used throughout this specification is, unless otherwise indicated, intended to encompass a hardware circuit (whether discrete elements or an integrated circuit block), a circuitry or control block executing code encoded in a computer readable media, or a combination of a hardware circuit(s) and a circuitry and/or control block executing such code.

All ranges and ratio limits disclosed in the specification and claims may be combined in any manner. Unless specifically stated otherwise, references to “a,” “an,” and/or “the” may include one or more than one, and that reference to an item in the singular may also include the item in the plural.

Although the inventions have been shown and described with respect to a certain embodiment or embodiments, equivalent alterations and modifications will occur to others skilled in the art upon the reading and understanding of this specification and the annexed drawings. In particular regard to the various functions performed by the above described elements (components, assemblies, devices, compositions, etc.), the terms (including a reference to a “means”) used to describe such elements are intended to correspond, unless otherwise indicated, to any element which performs the specified function of the described element (i.e., that is functionally equivalent), even though not structurally equivalent to the disclosed structure which performs the function in the herein illustrated exemplary embodiment or embodiments of the inventions. In addition, while a particular feature of the inventions may have been described above with respect to only one or more of several illustrated embodiments, such feature may be combined with one or more other features of the other embodiments, as may be desired and advantageous for any given or particular application.

1. A system for providing positive reinforcement on appropriate food items, the system comprising:
a storage unit for storing plurality of modules;
a graphical user interface for display processed plurality of modules;
a food central database to store type of nutrient and nutrient value associated with each food item;
a pillar database coupled to the food central database for categorizing the nutrients and the nutrient value of each food item into at least one of: a growth pillar; an energy pillar; an immunity pillar; and a detoxification pillar; and
a processing unit operably coupled to the food central database, the pillar database, the graphical user interface and to the storage unit to process the stored plurality of modules, wherein the plurality of modules comprising:
a growth module coupled to the pillar database to evaluate a growth score from the growth pillar for each food item, wherein the growth score is evaluated from the growth related nutrients value associated with each nutrient;
an energy module coupled to the pillar database to evaluate an energy score from the energy pillar for each food item, wherein the energy score is evaluated from the energy related nutrients value associated with each nutrient; and
an immunity module coupled to the pillar database to evaluate an immunity score from the immunity pillar for each food item, wherein the immunity score is evaluated from the immunity related nutrients value associated with each nutrient.

2. The system according to claim 1, wherein the plurality of modules further comprising a detoxification module coupled to the pillar database to evaluate a detoxification score from the detoxification pillar for each food item, wherein the detoxification score is evaluated from the detoxification related nutrients value associated with each nutrient.

3. The system according to claim 1, wherein the nutrient values are calculated from a pre-defined serving value.

4. The system according to claim 1, wherein the plurality of modules further comprising a registration module for registering users on receiving personal details, wherein the personal details comprising at least one of: a name; an address; health conditions; and a food preference.

5. The system according to claim 4. wherein the plurality of modules further comprising a food interest module for allowing registered users to input a list of food items through the graphical user interface.

6. The system according to claim 5, wherein the plurality of modules further comprising a food descriptive module for calculating and displaying score for each pillar based on the food items received in the food interest module.

7. The system according to claim 4, wherein the plurality of modules further comprising a food predictive module for evaluating and automatically suggesting the registered users to eat specific food items to improve on the growth score, the energy score, and the immunity score.

8. The system according to claim 4, wherein the plurality of modules further comprising a food prescriptive module for evaluating and automatically suggesting the food items to score better on the growth score, the energy score and the immunity score based on the health conditions received from the registered users.

9. The system according to claim 1, wherein the plurality of modules further comprising a search module to allow a registered user to search nutrients and nutrient value associated with each food item.

10. The system according to claim 4, wherein the plurality of modules further comprising a social media module allows the registered users to communicate with social media platforms.

11. A computer-implemented method for providing positive reinforcement on appropriate food items on a non-transitory computer-readable medium, wherein the method is performed at least in part by a processing unit, the method comprising:
storing plurality of modules using a storage unit;
displaying processed plurality of modules of a graphical user interface;
storing type of nutrient and nutrient value associated with each food item using a food central database;
categorizing the nutrients and the nutrient value of each food item into at least one of: growth pillar; energy pillar; immunity pillar; and detoxification pillar; using a pillar database coupled to the food central database; and
processing the stored plurality of modules using a processing unit operably coupled to the food central database, the pillar database and to the storage unit to, wherein the plurality of modules comprising:
a growth module coupled to the pillar database to evaluate a growth score from the growth pillar for each food item, wherein the growth score is evaluated from the growth related nutrients value associated with each nutrient;
an energy module coupled to the pillar database to evaluate an energy score from the energy pillar for each food item, wherein the energy score is evaluated from the energy related nutrients value associated with each nutrient; and
an immunity module coupled to the pillar database to evaluate an immunity score from the immunity pillar for each food item, wherein the immunity score is evaluated from the immunity related nutrients value associated with each nutrient.

12. The method according to claim 11, wherein the plurality of modules further comprising a detoxification module coupled to the pillar database to evaluate a detoxification score from the detoxification pillar for each food item, wherein the detoxification score is evaluated from the detoxification related nutrients value associated with each nutrient.

13. The method according to claim 11, wherein the nutrient values are calculated from a pre-defined serving value.

14. The method according to claim 11, wherein the plurality of modules further comprising a registration module for registering users on receiving personal details, wherein the personal details comprising at least one of: a name; an address; health conditions; and a food preference.

15. The method according to claim 14, wherein the plurality of modules further comprising a food Interest module for allowing registered users to input a list of food items through the graphical user interface.

16. The method according to claim 15, wherein the plurality of modules further comprising a food descriptive module for calculating and providing score for each pillar based on the food items received in the food interest module.

17. The method according to claim 14, wherein the plurality of modules further comprising a food predictive module for evaluating and automatically suggesting the registered users to eat specific food items to improve on the growth score, the energy score, and the immunity score.

18. The method according to claim 14, wherein the plurality of modules further comprising a food prescriptive module for evaluating and automatically suggesting the food items to score better on the growth score, the energy score and the immunity score based on the details received from the registered users.

19. The method according to claim 14, wherein the plurality of modules further comprising a search module to allow user to search nutrient value associated with each food item.

20. The method according to claim 14, wherein the plurality of modules further comprising social media module to link social media platforms with the plurality of modules.
